(19) United States
(12) Patent Application Publication
(10) Pub. No.: US 2007/0224668 A1
(43) Pub. Date: Sep. 27, 2007

(54) Process for producing 4-vinylguaiacol by biodecaroxylation of ferulic acid

(76) Inventors: Jianzhong Yang, Montreal (CA); Denis Rho, Montreal (CA); Peter C.K. Lau, Kirkland (CA); Kofi Abokitse, Saint-Laurent (CA)

Correspondence Address: GEORGE A. SEABY;SEABY & ASSOCIATES, 250 CITY CENTRE AVNUE, OTTAWA, ON, K1R6K7, US

(21) Appl. No.: 11/723,611

(22) Filed: Mar. 21, 2007

Related U.S. Application Data
(60) Provisional application No. 60/783,851, filed on Mar. 21, 2006.

(51) Int. Cl. C12P 7/42; C12N 9/88; C12N 15/74; C12N 1/21

(52) U.S. Cl. 435/146; 435/232; 435/252.33; 435/488

(57) ABSTRACT

4-vinylguaiacol is produced using recombinant E. coli containing a decarboxylase gene from Bacillus pumilis in an aqueous fermentation broth and in an immobilized whole cell system. The 4-vinylguaiacol is extracted and recovered from an organic hydrocarbon solvent, preferably n-octane, whereby the product can readily be separated.

### CROSS-REFERENCE TO RELATED APPLICATION

This application claims priority on U.S. Provisional Application 60/783,851 filed Mar. 21, 2006.

### BACKGROUND OF THE INVENTION

1. Field of the Invention

This invention relates to a process for producing 4-vinylguaiacol, and in particular to an integrated process for producing 4-vinylguaiacol by the biodecaroxylation of ferulic acid.

2. Description of Related Art

4-vinylguaiacol (VG) is a known flavour and fragrance compound which is generally regarded as safe. VG and other aroma compounds (guaiacol, vanillin) of natural origin are of great interest in the fragrance industry. Their use and application are well known to those of ordinary skill in the art. By using effective and balanced amounts of VG with other compounds, it is possible to augment or enhance the organoleptic properties of flavoured consumables, such as beverages, dairy products, baked goods and ice cream. VG produced by fermentation is especially valuable in any flavour composition where entirely natural ingredients are required. Although many natural products such as apple, grapefruit juice, strawberry, raw asparagus, stalks of celery, white and red wines, coffee, partially fermented tea, sesame seeds contain VG, nature alone cannot meet the ever-increasing world demand for the compound. Thus, because of their widespread applications in food and alcoholic beverages as well as intermediates in the preparation of biodegradable polymers and copolymers various research activities during the last decade have focused on the use of inexpensive and renewable crop residues for the production of natural aroma compounds and in particular, substituted 4-vinylphenols such as 4-vinylguaiacol, 4-hydroxystyrene and vanillin.

Ferulic acid (FA), which is abundantly available from different natural sources such as wood, flax shive, sugar beet molasses, corn bran, rice and wheat, is a starting material or substrate for biotransformation to 4-vinylguaiacol (VG). FA often occurs in the form of a glucoside in plant materials which can be isolated from corresponding glycosides in plants by well-known hydrolysis methods using enzymes and/or chemical processes. The FA can be used in crude or purified form. GB Patent Publication No. 2301103 A1 describes the enzymatic breakdown of ferulic acid containing plant material using a ferulic acid esterase to obtain the free acid. Other literature relating to biotransformation of FA include: P. N. Rosazza, B. Rousseau, Review: Biocatalytic Transformations of Ferulic Acid: An Abundant Aromatic Natural Product, J. Ind. Microbiol. 15 (1995) 457-471; P. A. Kroon, M. T. G. Williamson, Release of Ferulic Acid Dehydrodimers from Plant Cell Walls by Feruloyl Esterases, J. Sci. Food Agri. 79 (1999) 428-434; A. I. Sancho, C. B. Faulds, Release of Ferulic Acid from Cereal Residues by Barley Enzymatic Extracts, J. Cereal Sci. 34 (2001) 173-179; P. A. Kroon, G. Williamson, Release of Ferulic Acid from Sugar-Beet Pulp by using Arabinanase, Arabinofuranosidase and an Esterase from Aspergillus niger, Biotechnol. Appl. Biochem. 23 Part 3 (1996) 263-267; C. B. Faulds, G. Williamson, Release of Ferulic Acid from Wheat Bran by a Ferulic Acid Esterase (FAE-III) from Aspergillus niger, Appl. Microbiol. Biotechnol. 43 (1995) 1082-1087; and B. Bartolome, G. Williamson, Release of the Bioactive Compound, Ferulic Acid, from Malt Extracts, Biochem. Society Transactions 24 (1996) S379-S37.9.

In Canada, oilseed flax straw (1 Mt/year) is considered to be a residue. After the recovery of fiber from flax straw for producing cigarette paper, huge quantities of shive (>70% by weight of the straw) is available as a renewable resource. Useful chemicals can be separated and isolated from shive using physical and/or chemical processes. Specific compounds, such as ferulic acid (FA) is a useful starting material for the production of value added products such as 4-vinylguaiacol (VG).

There are different ferulic acid decarboxylases (FDCs) described in the literature, and most of them have been purified and their encoding gene identified and cloned. All bacterial FDC described were expressed using their native promoter; no inducer was need in most of the cases. The ferulic acid decarboxylase (FDC) of Bacillus pumilus PS231 was first described by [Zago et al Appl. Environ. Microbiol. 61 (1995) 4484-4486]. The encoding gene was isolated and identified to be located on a 1332 bp HindIII-XbaI fragment. This fragment was cloned in pUC19 and transformed into E. coli DH5α cells. The recombinant cell was used for expression of the decarboxylase. The activity obtained was quite similar to that of the wild type strain; however, the ferulic acid decarboxylase expressed in E. coli was described as being unstable; a large part of the activity was lost during purification. It is worth noting that instability due to purification is different from inherent instability.

Four bacterial phenolic acid decarboxylases (PAD) from Lactobacillus plantarum, Pediococcus pentosaceus, Bacillus subtilus and Bacillus pumilus ATCC 15884 were also cloned and expressed in E. coli TG1 [Barthelmebs, Divies et al Appl. Environ. Microbiol. (2001), 67(3) 1063-1069] the plasmid used was pJDC9, a pUC19 derivative. The four enzymes displayed 61% amino acid sequence identity and they exhibit different activities for ferulic and caffeic acid. The C-terminal of the four proteins was compared. The FDC from the two Bacillus pumilus strains PS231 and ATCC 15884 show a similarity of 98% (difference in four amino acids). In Saccharomyces cerevisiae, a phenylacrylic acid decarboxylase that confers resistance to cinnamic acid in this strain [Clausen, Lamb et al Gene (1994), 142(1), 107-112)] was described.

Furthermore, another fungal decarboxylase was identified in the wine Saccharomyces cerevisiae W3. The gene encoding this decarboxylase was used to transform the S. cerevisiae K9H14 strain lacking naturally the decarboxylase activity

A process was described in which the FDC enzyme (503 amino acids) was used to provide ferulic acid decarboxylases (Shoji et al, U.S. Pat. No. 5,955,137).

In the past 10 years, several methods for the microbial or enzymatic production of VG have been proposed. Such methods are described in the following:

U.S. Pat. No. 6,468,566 discloses a method for the preparation of 4-vinylguaiacol from ferulic acid using decarboxylase enzyme,

U.S. Pat. No. 5,235,507 discloses a method for the preparation of 4-vinylguaiacol by the microbial conversion of ferulic acid at a pH of more than 9,

J. Biotechnol., (2000), 80, 195-202 discloses a method for the decarboxylation of ferulic acid to produce 4-vinylguaiacol using Bacillus pumilus,

Enzyme Microbial Technol., (1998), 23, 261-266 discloses a method for preparing 4-vinylguaiacol by the decarboxylation of ferulic acid using Bacillus pumilus,

J. Fermentation Bioeng., (1996), 82(1), 46-50, discloses a method for the isolation of 4-vinylguaiacol from distilled and stored model solutions of “shochu”,

J. Biol. Chem., (1993), 268, 23954-23958 discloses a method for preparing 4-vinylguaiacol from ferulic acid by decarboxylation using Rhodotorula ruba,

Appl. Environ. Microbial., (1993), 59, 2244-2250 discloses a method for the production of 4-vinylguaiacol from ferulic acid by decarboxylation using Saccharomyces cerevisiae and Pseudomonas fluorescens.

Although the methods described in the above-listed references have proven to be useful, they have defects which prevent their commercial application. Microbiological transformation is a technique, which is generally known to be eco-friendly, with mild operating conditions. However, large amounts of VG are not easily produced.

One problem is the low production rate of biocatalysts. The growth rate of the wild bacterium B. pumilus is quite slow and recombinant E. coli expression is not stable (Appl. Envi Microbio. (1995), 61, 4484-4486).

A second problem is that the cellular toxicity of VG, which at concentrations of above 1 g/L prevents cell growth, resulting in a low reaction activity (Enzyme Microbial Technol., (1998), 23, 261-266).

A third problem is the instability of the biocatalyst during the biotransformation process. A variety of techniques have been proposed for maintaining the stability of the biocatalyst. Immobilization of microbial cells on water-insoluble supports and utilization of immobilized cells as the biocatalyst is an effective method of increasing the bio-stability, as recently described in WO 96/134971.

Thus, in spite of the efforts made to date, a need still exists for an efficient process for producing 4-vinylguaiacol. An object of the present invention is to provide a relatively efficient process for producing VG from FA using a recombinant biocatalyst, two-phase biotransformation and cell immobilization.

Another object of the invention is to provide immobilized microbial cells, which are catalytically active for use in the preparation of 4-vinylguaiacol. The entrapment method of immobilization is preferred because enzymatic activity is maintained. A catalyst is captured in beads, which have good mechanical strength and kinetics comparable to that of free cells, and the beads are formed of natural materials, preferably alginate which is easy to use and inexpensive.

### BRIEF SUMMARY OF THE INVENTION

Accordingly, the invention relates to a process for producing 4-vinylguaiacol comprising the steps of cultivating recombinant E. coli containing a decarboxylase gene from Bacillus pumilus (preferably strain AM670) in an aqueous fermentation broth; adding an organic solvent and a ferulic acid substrate to the fermentation broth whereby 4-vinylguaiacol is formed and accumulates in the organic solvent; and separating the 4-vinylguaiacol from the organic solvent.

The first step in the process of the present invention is the gene cloning and overexpression of decarboxylase from B. pumilus in an E. coli host. The desired characteristics for the recombinant E. coli are that (1) the growth rate should be fast, i.e. in hours rather than in days as required for the growth of the parent bacterium B. pumilus, (2) no inducer is required and expression efficiency is rapid and stable; and (3) bioconversion for the preparation of VG occurs in one step.

The selected solvent should be non-hazardous, inexpensive and have a good biocapability. The characteristics of the two-phase biotransformation system are possible avoidance of product inhibition, the production of VG in a high yield in one bioreactor, and the easy recovery of VG of high purity.

In greater detail, the microbiological process for producing VG in accordance with the present invention includes the steps of (a) cultivating the microorganism E. coli, preferably the bacterium E. coli JM 109 [pKFAD], in a nutrient-fermentation broth wherein, the cultivating period is 4-28 hours and preferably about 8-12 hours until the carbon source glucose is consumed, (b) adding an organic solvent selected from the group consisting of octane, cyclohexane, hexane, n-dodecane and n-hexadecane (preferably octane) at a ratio to the broth of 1:1 to 1:20 and (c) adding ferulic acid in an amount of about 5 to 25 g/L of fermentation broth, either continuously or batch-wise. After an biotransformation period of approximately 2 to 24 hours, the conversion of FA to VG is complete. The ferulic acid is consumed and about 3 to 10 g/L of the VG has accumulated in the organic solvent. The product is recovered from the organic solvent. Separation of the VG from the solvent is performed by evaporation. The VG may also be separated from the solvent by distillation.

The microbiological process for producing VG from ferulic acid occurs in accordance with the following biochemical pathway:

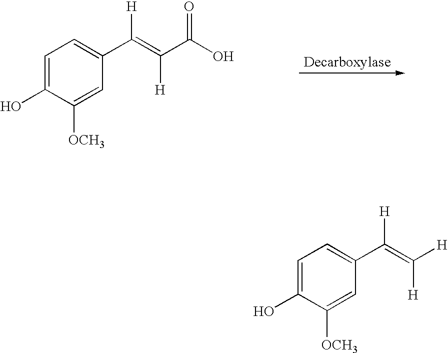

As pointed out above, exact fermentation conditions combined with an effective product recovery method result in a high yield of VG. The fermentation conditions are based upon the cultivation of the recombinant E. coli in an appropriate culture medium and the subsequent addition of an excess of ferulic acid about 5 to about 25 g/l to obtain VG at high volumetric yields in the organic phase. The preferred whole cell biocatalyst is E. coli JM 109.

The substrate, which is ferulic acid or a ferulic acid-containing compound is preferably trans-ferulic acid, namely 4-hydroxy-3-methoxycinamic acid.

In carrying out the present invention, cultivation of the bacterium is carried out in an aqueous medium in the presence of the usual nutrients. A suitable culture medium contains a carbon source, an organic or inorganic nitrogen source, inorganic salts and growth factors. Glucose is preferably used as the carbon source at a concentration of about 5-25 g/L, preferably about 10-20 g/L. Yeast extract, a useful source of nitrogen, phosphates, growth factors and trace elements may also be added. Magnesium sulfate is added at a concentration of about 0.1-5 g/L, preferably at about 0.5-1 g/L.

The culture broth is prepared and sterilized in a bioreactor, and is then inoculated with a preculture of recombinant E. coli at a ratio 1:10 in order to initiate the growth phase. An appropriate duration for the growth phase is about 4-48 hours, and preferably about 8-12 hours. The process conditions are a pH of 5 to 7 and a temperature of 7 to 37° C. Aeration and stirring are preferred.

At the end of the growth phase, an organic solvent and a ferulic acid substrate are added to the culture broth. A suitable amount of substrate is 5-25 g/L of the fermentation broth, preferably 10-20 g/L. The substrate is added either as a powder or as an aqueous solution. The total amount of substrate is fed in one step, in two or more steps or continuously. The biotransformation starts at the beginning of the substrate feed and lasts about 1-24 hours, preferably 2-8 hours until all of the FA substrate is converted to VG.

Since the biotransformation converts the hydrophilic substrate ferulic acid into hydrophobic VG, the overall volumetric productivity of the fermentation system is increased by applying an in-situ product recovery method. For this purpose, an extractive phase is added to the fermentation broth using a water-immiscible, organic solvent, preferably octane. Such an in-situ product recovery method allows continued formation of VG even after water soluble concentrations have been reached.

Upon completion of the biotransformation, organic solvent and the biomass in the aqueous phase are separated by any well known method, such as centrifugation, and the VG in the organic phase is further separated from the solvent by evaporation.

### BRIEF DESCRIPTION OF DRAWINGS

The process of the invention is described in greater detail with reference to the following examples, and the accompanying drawings, wherein:

FIG. 1 is a graph of VG production in various media;

FIG. 2 is a graph of VG production at various temperatures in an aqueous/organic system;

FIG. 3 is a graph of cell growth rate for B. pumilus;

FIG. 4 is a graph of VG production using FA induced B. pumilus;

FIG. 5 is a graph of recombinant E. coli cell growth rate;

FIG. 6 is a bar graph of FA biotransformations involving the multi-utilization of immobilized recombinant E. coli; and

FIGS. 7 and 8 are graphs illustrating the specific activity of E. coli cells in alginate beads.

### DETAILED DESCRIPTION OF THE INVENTION

### Example 1

### Biotransformation of Ferulic Acid using Wild Type B. pumilus as a Biocatalyst in a Mono-Aqueous Phase at Different Initial FA Concentrations

A pre-culture was prepared by inoculating colonies of Bacillus pumilus from agars in a Petri dish into a small flask containing 25 ml of the above described medium. Then 10 ml of the pre-culture was transferred into 100 ml of medium in a 500-mL Erlenmeyer flask containing Iowa medium (0.5 g/L ferulic acid, 20 g/L glucose, 5 g/L yeast extract, 5 g/L NaCl, 5 g/L tryptic soy broth, 5 g/L K₂ HPO₄.), or minimum medium or LB medium.

Standard culture conditions were as follows; temperature 30° C. and agitation rate 250 rpm. The pH was maintained at 6.8 by the addition of NaOH solution (1 M). Cell growth was observed by measuring cell concentration (optical density OD₆₀₀). Cells were harvested after 24 h of incubation by centrifugation (10000×g for 10 min). The resulting cell pellets were washed with 0.1 M phosphate buffer pH 6.8, then stored in ice for use as a biocatalyst for the biotransformation of ferulic acid.

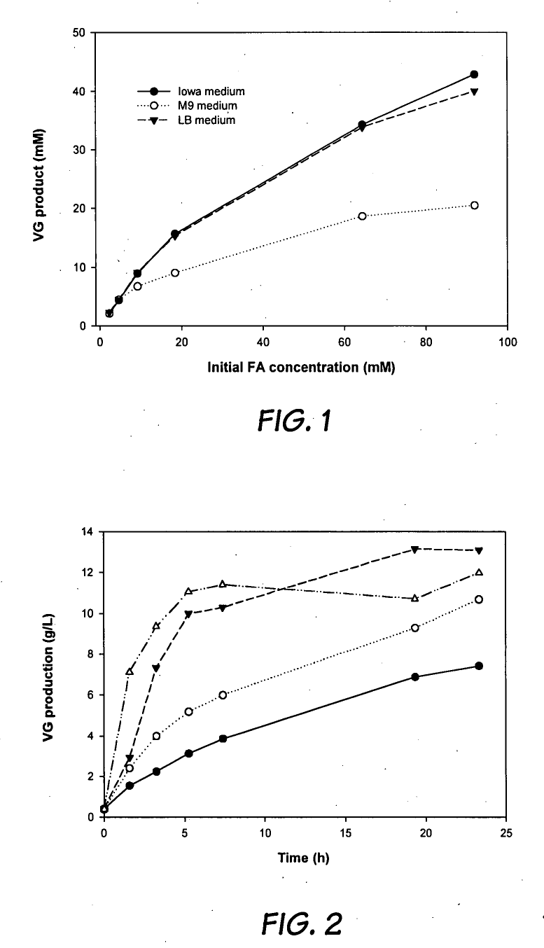

The biotransformation of the FA was performed in 20 ml bottles. The above described whole cell pellets were resuspended in 0.1 M phosphate buffer to a concentration of OD₆₀₀=10. Ferulic acid solution was added to the cell suspension for the biotransformation. The biotransformation was carried out at different initial FA concentrations. The experiments were performed at 30° C. for one hour with shaking at 250 rpm. To determine the reaction rate, reactions were stopped by adding 10 ml of 50% trichloroacetic acid to 1 ml of cells. Each reaction mixture was extracted using 9 volumes of methanol, centrifuged at 10,000×g for 10 min, and VG concentrations were determined by HPLC. The results are shown in FIG. 1. Cells cultured in Iowa and LB media (rich media) showed very similar activities. The activity of cells cultured in M9 minimal medium was significantly lower.

### Example 2

### Biotransformation of FA using Wild Type B. pumilus in an Organic Aqueous Two-Phase System

For whole cell biotransformations in a two-phase system, eight different solvents were selected for comparison purpose. The cells were resuspended in 1 ml of 0.1 M phosphate buffer to a concentration (OD₆₀₀=5) and mixed with an equal volume of organic solvent in flasks. Biotransformation was started at an initial FA concentration of 36 mM. The experiments were performed under the same conditions as in the mono-phase biotransformation process (Example 1). After stopping the reactions, reaction mixture (2 ml) was extracted with 18 ml of methanol. Considering the low solubility of dodecane and hexadecane in methanol, the organic phase was separated and analyzed using FTIR.

As illustrated in Table 1, two-phase bioconversion using non-polar hydrocarbons led to faster biotransformation (nearly 3 times higher activity than using water alone) and easier product recovery. Some polar solvents (ethanol, ethyl acetate) were toxic to the cells and resulted in low or no activity.

TABLE 1
Activity of resting cell in aqueous organic two-phase system, partition
coefficients or reactant and product in water and Log P values of solvents
in octanol/water

| | | Partition | | | |
|---|---|---|---|---|---|
| | | coefficient* | | Activity** | Log P*** |
| No | Solvent | FA | VG | μmol/min/g | octanol/water |
| 1 | Phosphate buffer | | | 51.4 | |
| | (Control) | | | | |
| 2 | Ethanol | | | 0.0 | −0.31 |
| 3 | Chloroform | 0.1 | 167 | 61.6 | 1.97 |
| 4 | Ethyl acetate | 0.4 | 120 | 3.0 | 0.73 |
| 5 | Cyclohexane C₆H₁₂ | <0.01 | 6 | 131.5 | 3.44 |
| 6 | n-Hexane C₆H₁₄ | <0.01 | 7 | 117.7 | 4.0 |
| 7 | n-Octane C₈H₁₈ | <0.01 | 6 | 129.2 | 5.15 |
| 8 | n-Dodecane C₁₂H₂₆ | <0.01 | <6 | 134.9 | 5.6 |
| 9 | n-Hexadecane | <0.01 | <6 | 120.9 | 8.25 |
| | C₁₆H₃₄ | | | | |

*Data from literature and experimental results.
**The inherent activity should be higher than those measured values, since the limitation of the substance at the end of reaction. New experiments were designed to get the inherent kinetic parameters.
***Calculated using Advanced Chemistry Development (ACD) Labs Software Solaris V4.67 (1994–2005/Labs) or Chemical Physics Handbood (1986).

### Example 3

### Temperature Effect on the Bioconversion using Wild Type B. pumilus

The effect of temperature on the reaction kinetics was determined under the same conditions. The initial reaction volume was 10 ml (5 ml cell suspension+5 ml octane). Samples were taken from the aqueous phase and the organic phase separately to follow the production rate and enzyme stability for 24 h.

The solubility of ferulic acid in the aqueous phase is significantly influenced by temperature. The reaction kinetics also depends on the temperature. Therefore, the productivity of VG is a function of temperature. Biotransformations were performed at four temperatures (7-37° C.) in an aqueous-octane (1:1) two-phase system and the results are shown in FIG. 2. In FIG. 2, [symbol] 7° C. [symbol] 15° C.; [symbol] 25° C.; [symbol] 37° C. Initial FA and cell concentrations in the aqueous phase were 25 g/l and 2.15 g DCW/L (dry cell weight per liter), respectively. According to the results, a higher reaction rate was observed at higher temperatures. Biotransformation at between 20 and 37° C. could be effected by maintaining a high reaction rate and long-term enzyme stability.

### Example 4

### Growth of Wide Type B. pumilus in Medium with FA is Slow but FA is Required as Inducer to Produce Active Biocatalyst

A pre-culture was prepared by inoculation of colonies (Bacillus pumilus) from agars in Petri dishes into small flasks containing 25 ml of the above described medium. Then 10 ml of pre-culture was added into 100 ml of medium in three 500-mL Erlenmeyer flasks containing Iowa medium (0.5 g/L ferulic acid, 20 g/L glucose, 5 g/L yeast extract, 5 g/L NaCl, 5 g/L tryptic soy broth, 5 g/L K₂ HPO₄.) or minimum medium with FA (0.5 g/L) or without FA.

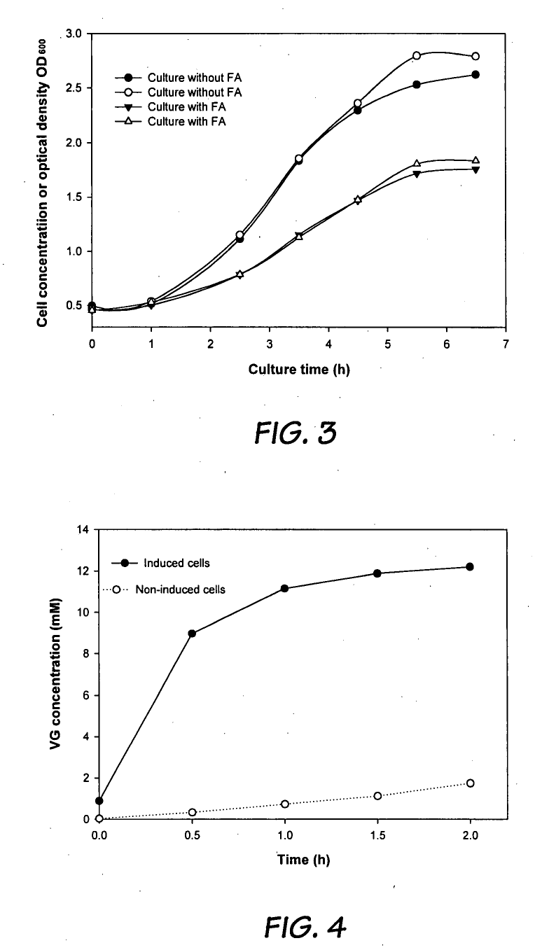

The culture conditions were as follows: temperature 30° C. and agitation 250 rpm. The pH was maintained at 6.8 by the addition of NaOH solution (1 M). Cell growth was observed by measuring cell concentration (optical density OD₆₀₀). Cells were harvested by centrifugation (10000×g for 10 min). The resulting cell pellets was washed with 0.1 M phosphate at a buffer of pH 6.8, then stored in ice as a biocatalyst for biotransformation of FA. When the FA was present in the cell culture, the growth rate is much lower than without FA (See FIG. 3).

When the harvested cells were used for the biotransformation of FA, the results indicated that the wild type B. pumilus needs to be induced using FA in the culture to obtain a high bioactivity (See FIG. 4). In order to obtain the results shown in FIG. 4, whole cells were induced using 0.5 g/L of FA in Iowa culture medium.

### Example 5

### Decarboxylase in Wild Type B. pumilus and in Recombinant E. coli JM109 Blank Experiments for Control

As described above, the Gene encoding for the Bacillus pumilus. AM 670 ferulic acid decarboxylase (fdc) was cloned into a commercially available pKK223-3 vector (sites PstI/HindIII). The 827 bp fragment containing the fdc coding sequence (486 bp) and the putative FDC native promoter (335 bp) was used. The recombinant pKFAD plasmid was transformed into E. coli JM109.

The nucleotide sequence and the corresponding amino acids sequences were published on the NCBI database under the accession number X84815.1 (Zago, Degrassi et al. 1995). The sequence of the cloned gene was identical to the sequence in the literature.

When an organic solvent, such as octane was used, the FA in the buffer and solvents without bacteria were examined, and no biotransformation was observed at 30° C., which is a blank control for solvents.

### Example 6

### Gene Clone of Decarboxylase from B. pumilus into E. coli Comparison of Growth Rate and Biotransformation Activity of E. coli with Wild Type B. pumilus in Bioreactor

In order to develop a process for bioconversion of ferulic acid into 4-vinylguaiacol, a biocatalyst consisting of a recombinant ferulic acid decarboxylase was designed. As described in Example 5, the gene encoding for the Bacillus pumilus AM670 ferulic acid decarboxylase (fdc) was cloned into the pKK223-3 vector (sites PstI/HindIII). The 827 bp fragment containing the fdc coding sequence (486 bp) and the putative FDC native promoter (335 bp) was used for this purpose. The recombinant pKFAD plasmid was transformed into E. coli JM109. The decarboxylase could be expressed after growing the cells at 30° C. overnight. The growth rate μ and the cell double time are 0.48 h⁻¹ and 1.44 h, respectively for E. coli. The high enzyme concentration in the whole cells resulted in a 10 times higher specific conversion rate (see Table 2). Using the new enzyme expression system at a cell concentration of 2.15 g DCW/L, the productivity could be increased from 2.6 to 26 g/h/L.

TABLE 2
Comparison of the over expression system with wild type bacteria

| | | Generation | Specific | Productivity |
|---|---|---|---|---|
| | Growth rate* | time | activity | at 2.15 g** |
| Biocatalysts | μ (h⁻¹) | (h) | (mmol/h/g) | (VG g/h/L) |
| B. pumilus | 0.21 | 3.15 | 6.9 | 2.6 |
| E. coli JM 109 | 0.48 | 1.44 | 69.8 | 26 |
| [pKFAD] | | | | |

*In LB culture medium with glucose
**The calculated values based on the bioactivity.

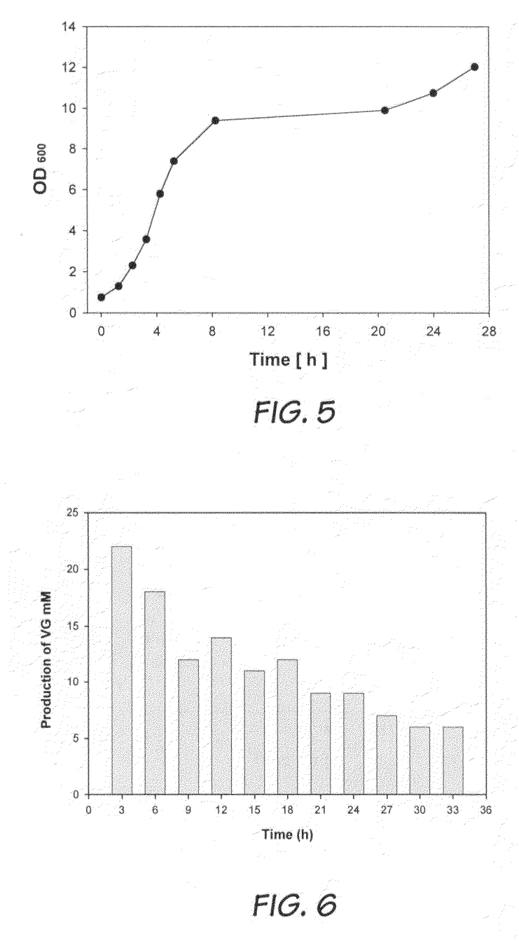

An important advantage for the new enzyme overexpression system is that it is constitutive, meaning that no induction by an otherwise expensive inducer, IPTG, is required. The enzyme expression is stable even after exponential growth phase (normally instability for the induction system is a problem during the enzyme expression). Such a system can ensure the quality of biocatalyst harvested at any time after the exponential growth phase or directly used in the bioreactor. FIG. 5 of the drawings shows the growth curve for the recombinant E. coli during a 28 h. period.

### Example 7

### Biotransformation using E. coli using Two-Phase ISPR in Bioreactor

A preculture of E. coli JM109 [pKADF] was grown in a shake flask at a pH 6.8, 30° C., 250 rpm, for 16 hours. The shake flask medium contained 10 g/L glucose in LB medium.

In a second experiment a 3 L bioreactor was filled with 900 ml of LB medium. After thermal sterilization, 20 g/L of sterilized glucose was added. Then the reactor was inoculated with 100 mL of the previously grown preculture. The process conditions were 30° C., pH 6.8, airflow rate 1.0 vvm, and 600 rpm. After 24 hours of growth, a remaining glucose concentration of 2 g/L was measured. Octane (250 ml) and 10 g of ferulic acid powder were added to the fermentation broth. After the addition of the FA precursor, the biotransformation of ferulic acid to VG was observed. The function of the octane in the bioreactor is to effect continuous and selective extraction of VG from the aqueous phase. The FA was not extracted into the organic phase and remained in the aqueous phase for further biotransformation. Ferulic acid was almost completely converted into VG as confirmed by HPLC analysis.

The organic phase was recovered and separated by centrifugation. A total volume of 230 ml octane containing VG was collected. Purification was effected by adding Na₂SO₄ (about 5 g) to remove (to chemically trap) the water, and then the octane was evaporated using a vacuum rotary evaporator. 4.65 g of VG were obtained with a purity of 97.5%. Overall, a VG recovery molar yield of 58.9% was calculated.

In a second experiment a 3 L bioreactor was filled with 900 ml of LB medium. After thermal sterilization, 20 g/L of sterilized glucose was added. Then the reactor was inoculated with 100 mL of the previously grown preculture. The process conditions were 30° C., pH 6.8, airflow rate 1.0 vvm, and 600 rpm. During the hour following 8.5 hours of growth 1000 ml of octane and 25 g of ferulic acid powder were added to the fermentation broth. Within the hour after the addition of the FA substrate, the almost complete biotransformation of ferulic acid to VG by HPLC was confirmed. The FA was not extracted into the organic phase, but remained in the aqueous phase for further biotransformation. Ferulic acid was almost completely converted into VG as confirmed by HPLC analysis.

The organic phase was recovered and separated by centrifugation. A total volume of 850 ml of octane containing VG was collected (the remaining 150 ml octane were left because they were trapped in a water-octane emulsion). Purification was effected by adding about 12 g of Na₂SO₄ to the octane to remove any remaining water. Then the octane was evaporated using a vacuum rotary evaporator. 13.8 g of VG were obtained with a purity of 98.4%. Overall, a VG recovery molar-yield of 68% was calculated.

### Example 8

### Cell Immobilization and Multi-Utilization of Biocatalyst for Biotransformation

E. coli JM109 [pKADF] cells were grown under standard fermentation conditions in a 3 L bioreactor with a 1000 ml working volume according to the procedure described in the previous example. The broth was centrifuged at 10,000×g for 10 minutes to yield a cell paste. About 12 grams of paste were obtained from 1000 ml of broth. The cell paste was conserved at −20° C. for use. An alginate (Protanal® GP4650, FMC Biopolymer) solution was prepared by adding 2.4 g of the alginate to 100 ml sterilized water. The cells paste (6.25 g) was suspended in 200 ml of phosphate buffer (0.1 M, pH 7). The cells in the phosphate buffer were mixed with the alginate solution (1/1 (v/v)). The alginate cell mixture was immediately pipetted dropwise (16 G) into a 2% CaCl₂ solution maintained at room temperature. The beads were gently agitated for 10 minutes to complete hardening and then were filtered from the CaCl₂ solution. The immobilized cells later showed a rate of 0.013 m mol VG produced per g dry cells per hour.

Multi-utilization of immobilized cells was tested. The half-life of activity, calculated as the time for the activity to reach 50% of the peak level, is estimated to be about 18 hours (see FIG. 4) The above data was obtained using batch reactions of 3 hours at 30° C. and 25 mM FA at a pH of 8.5. After 11 batches of bioconversion (33 h), 144 mM VG was produced using the same immobilized biocatalyst.

### Example 9

### Characterization for Immobilized Beads for Biotransformation

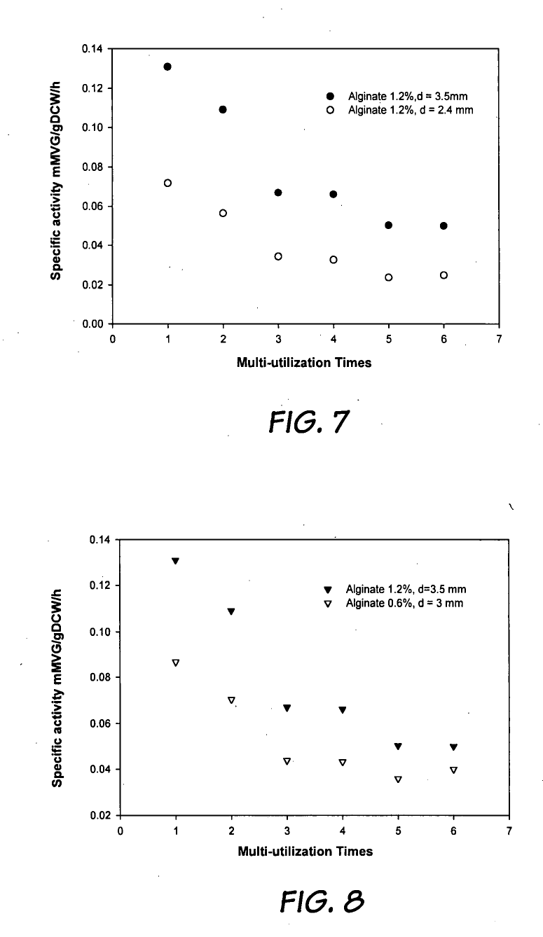

E. coli cells were prepared as indicated in Example 5. The cells in phosphate buffer were mixed with the alginate solution at different ratios. The alginate-cell mixture was immediately pipetted dropwise (16 gauge and 22 gauge) into a 2% CaCl₂ solution maintained at room temperature. The beads were gently agitated for 10 minutes to complete hardening and then were filtered from the CaCl₂ solution. The immobilized cells showed different specific activity [see FIGS. 7 and 8, which illustrate the effect of bead size (2.4 mm v. 3.5 mm diameter) and the effect of alginate concentration (0.6% v. 1.2%) on the biotransformation reaction rate]. Increases in bead size and alginate concentration resulted in high specific activity.

The advantages of the integrated bioprocess can be summarized as follows:
VG is produced using a recombinant microorganism, e.g. E. coli, which contains the genetic material coding for the enzymes involved in the cellular biosynthesis of VG.
the fermentation conditions enable the fast culturing of whole cell biocatalyst. The VG in the fermentation broth can reach economically attractive concentrations (about 3-10 g/L).
in situ product recovery techniques are used in a two-phase bioreactor system with an organic solvent, which is cheap and easily separated with the VG.
ferulic acid is one of raw materials which is available from easily accessible bioresources (plant residues).
cell immobilization is used to produce VG, which has the advantage of multiutilization (or continuous utilization) of biocatalysts in an economical fashion.

1. A process for producing 4-vinylguaiacol comprising the steps of cultivating recombinant E. coli containing a decarboxylase gene from Bacillus pumilus in an aqueous fermentation broth; adding an organic solvent and a ferulic acid substrate to the fermentation broth whereby 4-vinylguaiacol is formed and accumulates in the organic solvent; and separating the 4-vinylguaiacol from the organic solvent.

2. The process of claim 1, wherein the recombinant E. coli is added to the aqueous fermentation broth immobilized in aliginate.

3. The process of claim 1, wherein the E. coli is E. coli JM 109.

4. The process of claim 3, wherein the B. pumilus is strain AM670.

5. The process of claim 1, including the steps of preparing a recombinant E. coli cell paste; and combining the paste with alginate to produce beads of immobilized E. coli biocatalyst for use in the cultivating step of the process.

6. The process of claim 1, wherein the organic solvent is selected from the group consisting of cyclohexane, n-hexane, n-octane, n-dodecane and n-hexadecane.

7. The process of claim 1, wherein the organic solvent is n-octane.

8. The process of claim 1, wherein the recombinant E. coli is cultivated for 4 to 48 hours, the organic solvent is added to the fermentation broth at a ratio to the broth of 1:1 to 1:20, and the ferulic acid substrate is added to the broth in an amount of 5 to 25 g/L of broth.

9. The process of claim 8, wherein the fermentation is performed at a pH of 5 to 7 and a temperature of 7 to 37° C.

10. The process of claim 9, wherein the fermentation is performed at a temperature of 30° C.

11. The process of claim 5, wherein recombinant E. coli cells are grown in a fermentation broth, the broth is centrifuged to yield the cell paste, the cell paste is suspended in an alginate solution, and the mixture thus produced is added dropwise to calcium chloride solution to produce the beads.
